(19) United States
(12) Patent Application Publication
(10) Pub. No.: US 2015/0320505 A1
(43) Pub. Date: Nov. 12, 2015

(54) METERING OF A PREPARATION FLUID FOR SURGICAL INSTRUMENTS

(71) Applicant: OLYMPUS WINTER & IBE GMBH, Hamburg (DE)

(72) Inventor: Henning THATE, Hamburg (DE)

(73) Assignee: OLYMPUS WINTER & IBE GMBH, Hamburg (DE)

(21) Appl. No.: 14/805,893

(22) Filed: Jul. 22, 2015

Related U.S. Application Data
(63) Continuation of application No. PCT/EP2014/000116, filed on Jan. 16, 2014.

(30) Foreign Application Priority Data
Jan. 23, 2013 (DE) DE 102013201050.8

(51) Int. Cl. A61B 19/00; B08B 3/04

(52) U.S. Cl. CPC A61B 19/34; B08B 3/04; A61B 2019/343

(57) ABSTRACT

A metering apparatus for a reprocessing device for reprocessing surgical instruments. The metering apparatus including: a metering chamber for accommodating a reprocessing fluid, wherein the metering chamber comprises: an inlet for letting the reprocessing fluid into the metering chamber; and an outlet for letting out the reprocessing fluid introduced into the metering chamber, wherein the inlet and the outlet are dimensioned such that a flow velocity of the reprocessing fluid from the outlet while the reprocessing fluid is being let out of the metering chamber is greater than a flow velocity of the reprocessing fluid into the inlet while the reprocessing fluid is being introduced into the metering chamber.

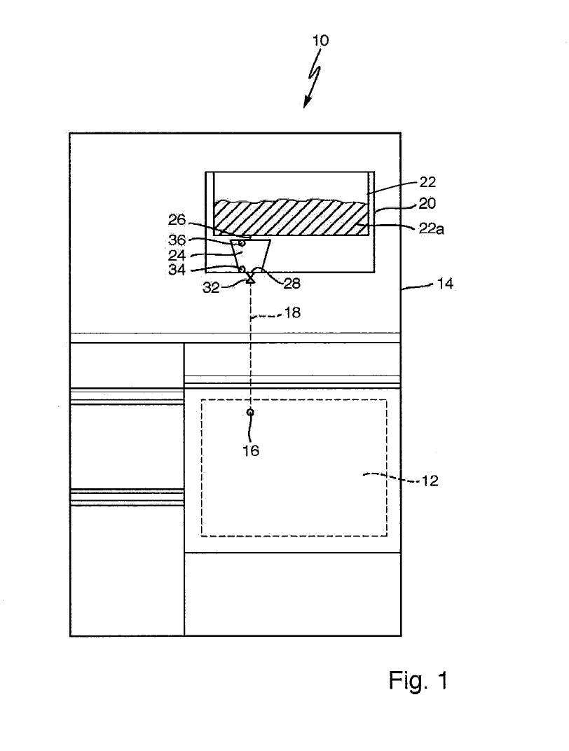

### CROSS-REFERENCE TO RELATED APPLICATION

The present application is a continuation of PCT/EP2014/000116 filed on Jan. 16, 2014, which is based upon and claims the benefit to DE 10 2013 201 050.8 filed on Jan. 23, 2013, the entire contents of each of which are incorporated herein by reference.

### BACKGROUND

1. Field

The present application relates to a metering apparatus for a reprocessing device for reprocessing surgical instruments, particularly endoscopes. The application further relates to a use of such a metering apparatus and a reprocessing device for reprocessing surgical instruments, particularly endoscopes, and a method for reprocessing a surgical instrument, particularly an endoscope, by means of a reprocessing fluid.

2. Prior Art

It is known in the state of the art that endoscopes are used to diagnose and treat diseases. For the use of endoscopes as well as other surgical devices, these surgical instruments must be reprocessed, i.e. cleaned and disinfected, after being used on a patient. In the case of the reprocessing of surgical instruments, legal and clinical regulations must be strictly adhered to so that the components inside and on the surface of the surgical instruments, in particular endoscopes, are disinfected and must be free of germs, bacteria, etc.

Reprocessing devices of surgical instruments, in particular of endoscopes, normally have devices which clean and disinfect the outer surfaces of the surgical instruments as well as the channels and channel systems using appropriate liquids of the same. Washing cycles are normally provided for this. In particular, in the case of the inner channel washing of a surgical instrument, in particular of an endoscope or respectively a flexible endoscope, the sanitary result of the reprocessing depends on the flow volume of a reprocessing fluid.

Moreover, a reprocessing device from Olympus Winter & Ibe GmbH, Hamburg, under the name ETD is known for reprocessing and disinfecting flexible endoscopes. In this device, the channels of the endoscopes are washed with a reprocessing fluid or respectively washing, cleaning and/or disinfecting liquids through mechanical cleaning and disinfection of, in particular, flexible endoscopes.

In the known reprocessing machines, hose pumps are used to meter the disinfecting agents and cleaning agents, wherein the hose pumps remove the reprocessing fluids from a canister or the like by means of a suction lance.

### SUMMARY

Based on this state of the art, an object is to enable a simple and maintenance-free metering of reprocessing fluids for cleaning and disinfecting surgical instruments, particularly endoscopes.

This object is solved by a metering apparatus for a reprocessing device for reprocessing surgical instruments, in particular endoscopes, with a predetermined metering chamber for accommodating a reprocessing fluid, particularly a cleaning liquid or a disinfecting liquid, wherein the metering chamber comprises an inlet for letting the reprocessing fluid into the metering chamber and an outlet for letting out a reprocessing fluid introduced into the metering chamber, wherein the inlet and the outlet are dimensioned in such a way that the flow velocity of the reprocessing fluid in the outlet while the reprocessing fluid is being let out of the metering chamber is greater than the flow velocity of the reprocessing fluid in the inlet while the reprocessing fluid is being introduced into the metering chamber.

The metering apparatus is based on the idea that the metering chamber of the metering apparatus has a predetermined volume, into which the reprocessing fluid is introduced from a reservoir, such as being above the metering chamber, as a result of a gravitational filling and via the inlet into the metering chamber, such as being below the reservoir, wherein in particular the filling time for filling the metering chambers is much greater than the time for emptying the metering chamber. The metering chamber is hereby filled slowly with the reprocessing fluid from the reservoir over a longer period of time up to the maximum fill level and then emptied, within a short period of time, particularly suddenly, due to a flow cross-section in the outlet that is greater with respect to the inlet.

According to the application, the flow velocity while the reprocessing fluid is being let out of the metering chamber is greater than the flow velocity of the reprocessing fluid during the filling of the metering chamber in the inlet, such as by a factor of 2, 3, 4, 5 to 10 or 20. Accordingly, the filling time, i.e. the time for filling the initially empty metering chamber up to the complete fill level is greater than the time for emptying the metering chamber via the outlet, such as at least by a factor of 2, 3, 4, 5 to 10 or respectively 20.

A metering of the reprocessing fluid is hereby provided in that the metering chamber is refilled significantly slower by the subsequent reprocessing fluid from the container than the extraction or respectively the emptying of the filled metering chamber through the opened outlet. If, for example, the extraction is faster than the filling by a factor of 100, a metering accuracy of 1% is achieved.

In particular, the inlet and the outlet are dimensioned in such a way that a metering accuracy of less than ±5% (<±5%) is set.

Another advantage is that, in a reprocessing device for surgical instruments, the use of hose pumps or other pump apparatuses for extraction of reprocessing fluids from containers is unnecessary so that a pump-free metering of reprocessing fluids for surgical instruments is provided.

Moreover, a further development is that a valve is provided on the outlet, wherein in particular the valve is closed during the filling of the metering chamber or is open during the letting of the reprocessing fluid out of the metering chamber.

Furthermore, the inlet can be designed as a valveless inlet, wherein the inlet is connected or connectable with the container filled with reprocessing fluid. Due to the fact that the reprocessing fluid flows out of the container via the inlet into the metering chamber due to gravity, a gravimetric metering system is provided. In such embodiment, the container is hereby provided with the reprocessing fluid above the metering chamber.

Moreover, one embodiment of the metering apparatus is characterized in that at least one control window is provided for the optical monitoring of the fill level of the reprocessing fluid in the metering chamber.

It is thereby also possible that a fill level display or a fill level sensor system is provided for the metering chamber or respectively the metering apparatus in order to display or respectively determine the fill level in the metering chamber. Such a control unit can be designed for example by means of a photo sensor or the like.

Moreover, it is provided in a further embodiment of the metering apparatus that two spaced control windows are provided on the metering chamber in relation to the flow direction of the reprocessing fluid between the, inlet and the outlet where the inlet can be an upper inlet and the outlet can be a lower outlet. An optical monitoring of whether the metering chamber is emptied completely during this emptying is hereby possible by means of a lower control window, which can be in the area of the outlet.

The inlet can be connected or connectable with a container for the reprocessing fluid, wherein in particular the reprocessing fluid is automatically introducible or introduced into the metering chamber from the container via the inlet through gravity. The outlet of the metering chamber is hereby connected with an inlet for a reprocessing fluid in a washing area of a reprocessing device.

The object is further solved through a use of a metering apparatus, as described above, for metered delivery of a reprocessing fluid, in particular a cleaning liquid or a disinfecting liquid, in the washing area of a reprocessing device for reprocessing surgical instruments, particularly endoscopes.

Moreover, the object is solved by a reprocessing device for reprocessing surgical instruments, particularly endoscopes, with a washing area for accommodating and reprocessing at least one surgical instrument, particularly an endoscope, and with a metering apparatus, as described above, for the metered delivery of a reprocessing fluid into the washing area. We expressly refer to the above explanations in order to avoid repetitions.

The object is further solved by a manageable reservoir, in particular canister, with a tank for a reprocessing fluid for reprocessing a surgical instrument, in particular endoscope, and with a previously described metering apparatus. For example, the reservoir is hereby provided with an integrated metering apparatus so that the metering chamber of the reservoir is attached to a reprocessing device at the outlet.

Simple handling is achieved through the provision of a reservoir with an integrated metering apparatus or respectively metering chamber, wherein upon replacement of an empty reservoir with a reservoir filled with a reprocessing fluid on the reprocessing device, the metering chamber is also replaced together with the reservoir so that the reservoir replacement results in the replacement of a used metering chamber of the empty reservoir with a sanitary metering chamber of the full reservoir. The sanitation for reprocessing surgical instruments and cleaning reprocessing devices as well as their maintenance is thus improved in a simple and efficient manner.

The object is further solved by a method for reprocessing a surgical instrument, particularly an endoscope, by means of a reprocessing fluid in a washing area, such as in a washing area of a reprocessing device, wherein upon connection of a metering apparatus, as described above, the reprocessing fluid is delivered in a metered manner to the washing area of the reprocessing device.

Further characteristics will become evident from the description of the embodiments together with the claims and the attached FIGURE. Embodiments can fulfill individual characteristics or a combination of several characteristics.

### BRIEF DESCRIPTION OF THE DRAWINGS

The embodiments are described below, without restricting the general idea of the invention, based on an exemplary embodiment in reference to the FIGURE, whereby we expressly refer to the FIGURE with regard to the disclosure of all details according to the invention that are not explained in greater detail in the text.

The FIGURE schematically illustrates a view of a reprocessing device for reprocessing surgical instruments, in particular endoscopes.

### DETAILED DESCRIPTION

The FIGURE shows schematically a reprocessing device 10 for endoscopes in a front view. For reprocessing surgical instruments, the reprocessing device thereby has a housing 14 with a receiving area 12 designed inside the housing 14. The surgical instruments, e.g. endoscopes, to be cleaned are introduced e.g. in baskets into the receiving area 12 designed as a washing area. An endoscope reprocessing device is known for example under the name ETD from Olympus Winter & Ibe GmbH, Hamburg.

The reprocessing device 10 also has a control and display panel as well as a drying device for the hot-air drying of surgical instruments.

Furthermore, the receiving area 12 has an inlet 16, which is connected with a reservoir 20 via a line 18.

The reservoir 20 itself is arranged above the receiving area 12 in a corresponding compartment or the like or holder. In this application, the terms “above” and “below” as well as similar terms thereto are relative to the direction of gravity, i.e., “above” meaning a direction higher than “below” in the direction of gravity, such that liquid would flow from the higher position to the lower position due to gravity. The reservoir 20 is further designed with a tank 22 for accommodating a reprocessing fluid 22a. For example, a cleaning liquid or a disinfecting liquid as well as another washing liquid in concentrated form can hereby be accommodated as the reprocessing fluid.

Moreover, the reservoir 20 is designed with a metering chamber 24, wherein the metering chamber 24 is connected with the tank 22 via an inlet 26. The metering chamber 24 has a volume that matches the metering amount of reprocessing liquid, which is introduced in concentrated form into the receiving area 12 via the inlet 16. The tank 22 is thereby positioned above the inlet 26 and the metering chamber 24.

Due to gravity, the reprocessing liquid from the tank 22 is introduced into the metering chamber 24 via the inlet 26 so that the metering chamber 24 is fillable with the reprocessing fluid. The metering chamber 24 has on its bottom side an outlet 28 connected with the line 18, via which the reprocessing fluid drains via the outlet 28 via the line 18 connected to the outlet 28 after the filling of the metering chamber 24 with the reprocessing fluid. A valve 32 is hereby arranged on the outlet 28.

The upper inlet 26 and the lower outlet 28 are designed such that the flow velocity of the reprocessing fluid in the inlet 26 is much smaller than during the letting of the reprocessing fluid out of the filled metering chamber 24, whereby the reprocessing fluid flows slowly into the metering chamber 24 from the tank 22 via the inlet 26 to fill the metering chamber 24, while the reprocessing fluid flows faster out of the metering chamber 24 via the outlet 28 after the filling of the metering chamber 24, whereby a predetermined amount of reprocessing fluid flows into the receiving area 12 via the line 18 and the inlet 16.

For monitoring the fill level of the metering chamber 24 during the filling of the metering chamber 24, a control window 34, 36 is arranged in a lower area as well as in an upper area of the metering chamber 24. The fill level of the reprocessing fluid in the metering chamber 24 can hereby be determined visually.

All named characteristics, including those taken from the FIGURE alone, and individual characteristics, which are disclosed in combination with other characteristics, are considered alone and in combination as essential for the invention. Embodiments can be realized by individual characteristics, or a combination of several characteristics.

### LIST OF REFERENCE NUMBERS

10 Reprocessing device
12 Receiving area
14 Housing
16 Inlet
18 Line
20 Reservoir
22 Tank
24 Metering chamber
26 Inlet
28 Outlet
32 Valve
34 Control window
36 Control window

What is claimed is:

1. A metering apparatus for a reprocessing device for reprocessing surgical instruments , the metering apparatus comprising:
a metering chamber for accommodating a reprocessing fluid, wherein the metering chamber comprises:
an inlet for letting the reprocessing fluid into the metering chamber; and
an outlet for letting out the reprocessing fluid introduced into the metering chamber,
wherein the inlet and the outlet are dimensioned such that a flow velocity of the reprocessing fluid from the outlet while the reprocessing fluid is being let out of the metering chamber is greater than a flow velocity of the reprocessing fluid into the inlet while the reprocessing fluid is being introduced into the metering chamber.

2. The metering apparatus according to claim 1, further comprising a valve provided on the outlet, wherein the valve is one of closed during the filling of the metering chamber or is open during the letting of the reprocessing fluid out of the metering chamber.

3. The metering apparatus according to claim 1, wherein the inlet is a valveless inlet.

4. The metering apparatus according to claim 1, further comprising at least one control window provided on the metering chamber for the optical monitoring of the fill level of the reprocessing fluid in the metering chamber.

5. The metering apparatus according to claim 4, wherein the at least one control window comprises two control windows provided on the metering chamber, the two control windows being spaced in relation to a flow direction of the reprocessing fluid between the inlet and the outlet.

6. The metering apparatus according to one claim 1, wherein the inlet is connected with a container for the reprocessing fluid, wherein the reprocessing fluid is automatically introduced into the metering chamber from the container via the inlet through gravity.

7. A method for metered delivery of a reprocessing fluid in a washing area of a reprocessing device for reprocessing surgical instruments, the method comprising:
providing a metering chamber for accommodating the reprocessing fluid, wherein the metering chamber comprising an inlet for letting the reprocessing fluid into the metering chamber and an outlet for letting out the reprocessing fluid introduced into the metering chamber, and
dimensioning the inlet and the outlet such that a flow velocity of the reprocessing fluid from the outlet while the reprocessing fluid is being let out of the metering chamber is greater than a flow velocity of the reprocessing fluid into the inlet while the reprocessing fluid is being introduced into the metering chamber.

8. There method according to claim 7, further comprising delivering the reprocessing fluid from the outlet to the washing area in a metered manner.

9. A reprocessing device for reprocessing surgical instruments, the reprocessing device comprising:
a washing area for accommodating and reprocessing at least one surgical instrument; and
a metering apparatus comprising:
a metering chamber for accommodating a reprocessing fluid, wherein the metering chamber comprising an inlet for letting the reprocessing fluid into the metering chamber and an outlet for letting out the reprocessing fluid introduced into the metering chamber,
wherein the inlet and the outlet are dimensioned such that a flow velocity of the reprocessing fluid from the outlet while the reprocessing fluid is being let out of the metering chamber is greater than a flow velocity of the reprocessing fluid into the inlet while the reprocessing fluid is being introduced into the metering chamber.

10. A reservoir comprising:
a tank for a reprocessing fluid for reprocessing a surgical instrument; and
a metering apparatus comprising:
a metering chamber for accommodating a reprocessing fluid, wherein the metering chamber comprising an inlet for letting the reprocessing fluid into the metering chamber and an outlet for letting out the reprocessing fluid introduced into the metering chamber,
wherein the inlet and the outlet are dimensioned such that a flow velocity of the reprocessing fluid from the outlet while the reprocessing fluid is being let out of the metering chamber is greater than a flow velocity of the reprocessing fluid into the inlet while the reprocessing fluid is being introduced into the metering chamber.
